(19) United States
(12) Patent Application Publication
(10) Pub. No.: US 2012/0215243 A1
(43) Pub. Date: Aug. 23, 2012

(54) ULTRASONIC SURGICAL APPARATUS

(76) Inventors: Takamichi FUJII, Ashigarakami-gun (JP); Yasutoshi HIRABAYASHI, Ashigarakami-gun (JP)

(21) Appl. No.: 13/359,048

(22) Filed: Jan. 26, 2012

(30) Foreign Application Priority Data
Feb. 22, 2011 (JP) 2011-035820

(51) Int. Cl. A61B 17/32

(52) U.S. Cl. 606/169

(57) ABSTRACT

An ultrasonic surgical apparatus includes a treatment portion, a driving portion which drives the treatment portion by resonance, an operation portion main body which drives and controls the driving portion, and a connecting portion which connects the treatment portion and the operation portion main body. The driving portion includes a piezoelectric film and is formed on the treatment portion inside the connecting portion. The piezoelectric film has a perovskite structure, with crystals constituting the perovskite structure being oriented in the (100) direction or the (001) direction with a degree of orientation of not less than 60%.

### BACKGROUND OF THE INVENTION

1. Field of the Invention

The present invention relates to an ultrasonic surgical apparatus and, more particularly, to an ultrasonic surgical apparatus which can be used at a low driving voltage.

2. Description of the Related Art

Conventionally, there has been available treatment called ESD (Endoscopic Submucosal Dissection) which, when a polyp or early cancer is found in the stomach or the intestine, for example, the large or small intestine, locally excises a cancer tissue such as a polyp, early stomach cancer, large intestine cancer, or small intestine cancer by using an endoscope with minimum damage to the living body. In this ESD treatment, the operator marks a region, of the membrane, which is to be excised, and inflates the region of the morbid membrane by local injection. The operator then incises the membrane along the marked area by using a high-frequency treatment device (ultrasonic treatment device), and cuts the fibers constituting the submucosal layer to separate the membrane from the muscle layer.

Patent literature 1 (Japanese Patent Application Laid-Open No. 61-279239) discloses, as such an ultrasonic treatment device, an ultrasonic surgical apparatus which transmits ultrasonic waves to a treatment device upon increasing their amplitude with a horn using a Langevin transducer. Patent literature 2 (Japanese Patent Application Laid-Open No. 2002-186627) discloses an ultrasonic surgical apparatus which transmits the ultrasonic vibrations generated by an ultrasonic transducer to a horn, transmits the ultrasonic vibrations amplified by the horn to a probe, and then transmits the vibrations to a fixed blade on the tip of the probe.

Non-patent literature 1 (Minoru Kurosawa and Takeshi Sasanuma, “Enhancement of Vibration Speed of Micro Ultrasonic Scalpel using PZT Film”, The Institute of Electronics, Technical Report of IEICE, US2009-109 (213) 31) discloses a technique of applying the piezoelectric film formed by hydrothermal synthesis to an ultrasonic scalpel.

### SUMMARY OF THE INVENTION

The ultrasonic treatment device disclosed in the patent literatures 1 and 2 each use a Langevin transducer, which is obtained by stacking bulk piezoelectric materials. Such an arrangement leads to an increase in the size of a piezoelectric film. This makes it impossible to directly insert the Langevin transducer into the body. For this reason, the Langevin transducer is provided outside the body, and is designed to transmit ultrasonic waves to an affected area through a horn on its tip, thereby performing a treatment. In order to linearly transmit vibrations to an affected area by using the horn, the treatment device needs to have a linear structure. A further improvement has therefore been demanded in this device.

In addition, the device using the piezoelectric film as a material which is formed by hydrothermal synthesis according to the non-patent literature 1 (Minoru Kurosawa and Takeshi Sasanuma, “Enhancement of Vibration Speed of transducer for Micro Ultrasonic Scalpel using PZT Film”, The Institute of Electronics, Technical Report of IEICE, US2009-109 (213) 31) has not obtained satisfactory performance. In order to achieve a vibration speed of 7 msec, which is sufficient performance for a scalpel, it is necessary to set the driving voltage to 40 V or more. In consideration of safety, it is desired to set a low driving voltage for driving operation in the body.

The present invention has been made in consideration of the above circumstances, and has as its object to provide an ultrasonic surgical apparatus which is compact and lightweight, can be driven at a low driving voltage, and ensures safety even when being directly vibrated in the body.

In order to achieve the above object of the present invention, there is provided an ultrasonic surgical apparatus comprising a treatment portion, a driving portion which drives the treatment portion by resonance, an operation portion main body which drives and controls the driving portion, and a connecting portion which connects the treatment portion and the operation portion main body, wherein the driving portion comprises a piezoelectric film and is formed on the treatment portion inside the connecting portion, and the piezoelectric film has a perovskite structure, with crystals constituting the perovskite structure being oriented in one of a (100) direction and a (001) direction with a degree of orientation of not less than 60%. Please note that the expression such as (100) or (001) is a lattice plane of crystal expressed by Miller index.

According to the present invention, the piezoelectric film is used as the driving portion which drives the treatment portion, and the crystal structure forming the piezoelectric film is oriented in the (100) direction or the (001) direction with a degree of orientation of 80% or more. Since a piezoelectric constant d₃₁ (pm/V) can be improved by increasing the degree of orientation, the treatment portion can be driven at a low driving voltage. Driving the treatment portion at a low voltage allows to provide the driving portion on a portion inserted into the living body. This can increase the degree of freedom in the design of a structure which connects the apparatus main body and the treatment portion.

In the present invention, the piezoelectric film preferably has a dielectric loss of not more than 0.4.

According to the present invention, setting the dielectric loss of the piezoelectric film to 0.4 or less can suppress heat generation. This makes it possible to safely operate the apparatus even if the driving portion is provided on the portion inserted into the body.

In the present invention, it is preferable that the piezoelectric film has a columnar structure, with crystal grains being not more than 1 μm.

According to the present invention, forming the piezoelectric film into the columnar structure can make the vibration direction coincide with a direction perpendicular to the columns. This can improve the durability. In addition, since the material is displaced in one direction, heat generation can be suppressed as compared with a piezoelectric film having a random structure. Note that “crystal grains being not more than 1 μm” indicates that the crystal grains may include crystal grains of 1 μm or more as long as crystal grains of 1 μm or less are dominant grains. Crystal grains of 1 μm or less preferably occupy 60% of the overall crystal grains.

In the present invention, it is preferable that the piezoelectric film is formed on the treatment portion through a lower electrode, and the lower electrode and the treatment portion are formed from different materials.

According to the present invention, the piezoelectric film is connected to the treatment portion through the lower electrode, and different materials are used for the lower electrode and the treatment portion. Providing the lower electrode can prevent the formation of an oxide film between the piezoelectric film and the treatment portion. Therefore, this can prevent an increase in driving voltage.

In the present invention, the lower electrode preferably comprises a noble metal.

According to the present invention, using a noble metal as the lower electrode can prevent oxidation. This can prevent an increase in resistance due to oxidation, and hence can prevent an increase in driving voltage.

In the present invention, the piezoelectric film preferably has a thickness of not more than 10 μm.

According to the present invention, the thickness of the piezoelectric film is 10 μm or less. A decrease in the thickness of the piezoelectric film can achieve a decrease in driving voltage.

In the present invention, the piezoelectric film preferably comprises a perovskite-type oxide including one of lead zirconate titanate (PZT) and sodium potassium niobate (KNN).

The present invention defines materials for the piezoelectric film, and allows to form a film having good piezoelectric performance by using the above materials.

In the present invention, it is preferable to further comprise an upper electrode on the piezoelectric film and a resin which covers the upper electrode or the upper electrode and the piezoelectric film.

According to the present invention, covering the upper electrode can prevent electric shock when the upper electrode is inserted into the living body. In addition, when a lead material for the piezoelectric film is used, it is preferable to cover the piezoelectric film with a resin.

In the present invention, it is preferable that the piezoelectric films are formed on two surfaces of the treatment portion, and a vibration speed obtained by applying a driving voltage of 15 V to the piezoelectric films is not less than 8 m/s.

According to the present invention, forming the piezoelectric films on the two surfaces can form a surgical apparatus which operates at a low driving voltage and achieve a vibration speed of 8 m/s with satisfactory performance.

In the present invention, it is preferable that the treatment portion gradually decreases in width from a portion on which the driving portion is formed to a tip of the treatment portion.

According to the present invention, since the treatment portion gradually decreases in width from the portion on which the driving portion is formed to the tip of the treatment portion, it is possible to increase the vibration speed of the tip of the treatment portion.

According to the ultrasonic surgical apparatus of the present invention, forming the driving portion by using the piezoelectric film, it is possible to achieve reductions in size and weight. In addition, since the treatment portion can be operated at a low driving voltage, the treatment portion can be safely used even if the driving portion is inserted into the body and directly vibrated.

### BRIEF DESCRIPTION OF THE DRAWINGS

FIG. 1 is a view showing the overall arrangement of an ultrasonic surgical apparatus;

FIG. 2 is a schematic sectional view of a sputtering apparatus; and

FIG. 3 is a graph showing the relationship between the degree of orientation and a piezoelectric constant d₃₁.

### DETAILED DESCRIPTION OF THE PREFERRED EMBODIMENTS

A preferred embodiment of an ultrasonic surgical apparatus according to the present invention will be described below with reference to the accompanying drawings.

### [Overall Arrangement of Ultrasonic Surgical Apparatus]

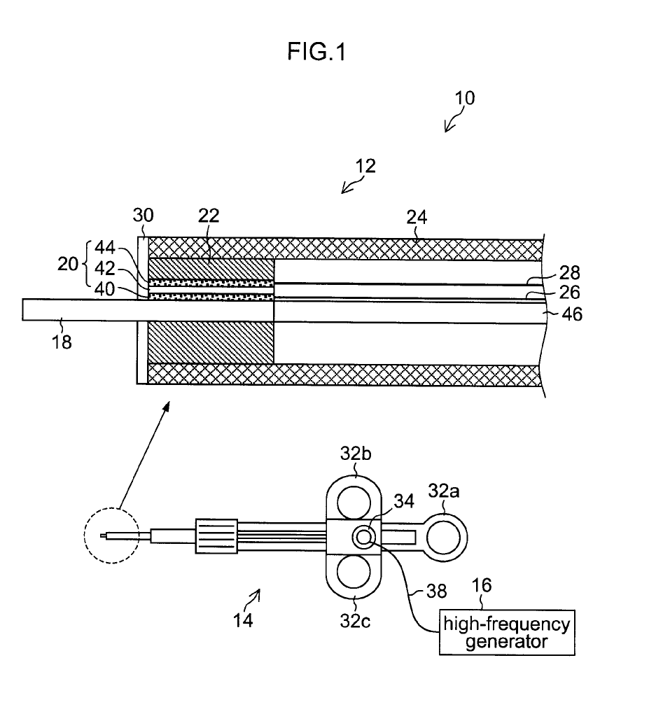

FIG. 1 is a view showing the overall arrangement of an ultrasonic surgical apparatus 10 of the present invention. The ultrasonic surgical apparatus 10 of the present invention includes a knife portion 12 functioning as an ultrasonic knife (scalpel) such as a needle-knife or a knife for peripheral incision and membrane separation (to be also referred to as a “dissection knife” hereinafter) in ESD treatment and an operation unit main body 14 which is operated by the operator to make the knife portion 12 function as an ultrasonic knife. The ultrasonic surgical apparatus 10 also includes a high-frequency generator 16 which applies a high-frequency voltage to the knife portion 12.

The knife portion 12 includes a blade portion (treatment portion) 18, a piezoelectric element 20, a fixed portion 22, a sheath (connecting portion) 24 having flexibility, a first electrode (ground potential) 26, a second electrode 28, a resin sealing member 30, and a flexible cord 46. The piezoelectric element 20 includes a lower electrode 40, a piezoelectric film 42, and an upper electrode 44.

The operation unit main body 14 includes rings 32a, 32b, and 32c for the operation of the blade portion 18 and a connector 34 as a connecting terminal for the high-frequency generator 16.

Note that the connector 34 of the operation unit main body 14 is electrically connected to the high-frequency generator 16 through a cord 38.

The blade portion 18 of the knife portion 12 functions as a dissection knife used for peripheral incision, round incision (cut), and submucosal dissection in ESD treatment, and is configured to vibrate by the vibration of the piezoelectric element 20. A material generally used for the blade portion 18 is a metal-based material such as titanium, a titanium alloy, stainless steel, duralumin, or an Ni—Cr—Mo—V-based refractory alloy steel. It is also possible to use a cemented ceramic material or the like.

The width of the blade portion 18 may gradually decrease from the portion where the piezoelectric element 20 is formed to the tip so as to have a horn portion. Providing the horn portion can increase the vibration speed of the tip of the blade portion 18, and hence can achieve a desired vibration speed at a low driving voltage. The vibration speed of the tip of the blade portion 18 can be determined by the transformation ratio which is the ratio between the width of the portion where the piezoelectric element 20 is formed and the width of the tip. It is possible to increase the vibration speed by increasing the transformation ratio (decreasing the width of the tip).

The piezoelectric element 20 includes the lower electrode 40, the piezoelectric film 42, and an upper electrode 44. The first electrode 26 connected to the lower electrode 40 and the second electrode 28 connected to the upper electrode 44 increase and decrease the strength of an electric field applied to the piezoelectric element 20 to expand and contract the piezoelectric element 20, thereby making the blade portion 18 ultrasonically vibrate in the direction indicated by the arrow shown in FIG. 1. This makes it possible to perform incision. Note that the lower electrode 40 may be provided as needed. If the lower electrode 40 is not provided, connecting the first electrode 26 to the blade portion 18 will apply a voltage to the piezoelectric element 20.

The fixed portion 22 is fixed to the inside of the distal end of the sheath 24, and has a function of supporting the blade portion 18 so as to allow the blade portion 18 to reciprocally move (move forward and retreat). That is, when the blade portion 18 protrudes and retreats from and into the distal end of the sheath 24, the fixed portion 22 supports the blade portion 18 so as to allow the blade portion 18 to move forward and retreat with respect to the sheath 24.

The sheath 24 is made of an insulating material having flexibility and physically and electrically protects the blade portion 18, the piezoelectric element 20, the first electrode 26, and the second electrode 28.

The first electrode 26 and the second electrode 28 serve to apply a high-frequency voltage to the piezoelectric element 20. These electrodes are made of a conductive material and respectively coupled to the rings 32b and 32c.

The resin sealing member 30 is provided to seal the end of the sheath 24 which is located on the living body side. In the present invention, the piezoelectric element 20 can be provided in a portion which is inserted into the body, and hence is preferably covered with a resin to prevent electric shock. In addition, since the piezoelectric film 42 can be made of lead, the piezoelectric film 42 is preferably covered with a resin. Using a resin as a sealing material for the sheath 24 can reduce the influence of resonance frequencies at the time of driving of the blade portion 18.

The arrangement and function of the ultrasonic surgical apparatus 10, in particular, the arrangement and function of the operation unit main body 14, will be described next by describing the operation method of the ultrasonic surgical apparatus 10 shown in FIG. 1.

The operator inserts his/her thumb, index finger, and middle finger into the rings 32a, 32b, and 32c of the operation slider, respectively, and slides the operation slider along the operation unit main body 14. With this sliding operation, the blade portion 18 can move forward and retreat (reciprocally move) from and into the sheath 24 through the flexible cord 46 coupled to the operation slider.

The high-frequency voltage cord 38 from the high-frequency generator 16 is connected to the connector 34, and the first and second electrodes 26 and 28 are electrically connected to the connector 34. Therefore, this high-frequency voltage is applied to both the first and second electrodes 26 and 28 to vibrate the piezoelectric element 20. This makes the blade portion 18 ultrasonically vibrate and makes it function as a dissection knife.

### [Piezoelectric Element]

The piezoelectric element of the present invention will be described next. According to the ultrasonic surgical apparatus 10 of the present invention, the piezoelectric element 20 as a driving portion is integrally provided for the blade portion 18. The piezoelectric element 20 includes the lower electrode 40, the piezoelectric film 42, and the upper electrode 44.

The lower electrode 40 can be provided as needed. If the lower electrode 40 is not provided, the first electrode 26 is directly grounded on the blade portion 18 to apply a voltage to the piezoelectric element 20. Providing the lower electrode 40 can prevent the formation of an oxide on the boundary between the piezoelectric film 42 and the blade portion 18, and hence can prevent an increase in driving voltage. According to the conventional direct growth method by hydrothermal synthesis, since a piezoelectric film is directly formed on Ti metal, an oxide is formed on the boundary between the piezoelectric film and the scalpel main body. This may increase the required driving voltage. For this reason, a material for the lower electrode 40 is preferably different from that for the blade portion 18, and is a noble metal resistant to oxidation. For example, this material is preferably a noble metal resistant to oxidation (Pt, Ir, Ru, or Au) or a noble metal having low resistance even after oxidation (Ir or Ru).

The composition of the upper electrode 44 is not specifically limited, and may include the materials exemplified above as those for the lower electrode 40, that is, electrode materials used for a general semiconductor process, such as Al, Ta, Cr, and Cu, and combinations of them.

The piezoelectric film 42 preferably contains, as a major component or components, one or two types of perovskite-type oxides represented by the following general formula (P1):

general formula ABO₃ (P1)

(A: A-site element including at least one kind of element selected from the group consisting of Pb, Ba, Sr, Bi, Li, Na, Ca, Cd, Mg, K, and lanthanide elements; B: B-site element including at least one kind of element selected from the group consisting of Ti, Zr, V, Nb, Ta, Cr, Mo, W, Mn, Mg, Sc, Co, Cu, In, Sn, Ga, Zn, Cd, Fe, Ni, Hf, and Al; and O: oxygen, although the standard molar ratios between A-site element, B-site element, and oxygen element are 1:1:3, the molar ratios may deviate from the reference molar ratios within a range in which perovskite structures can be formed)

The perovskite-type oxides represented by the above general formula (P1) include lead titanate, lead zirconate titanate (PZT), lead zirconate, and niobate-lead zirconate titanate. A piezoelectric film may be a mixed crystal of these perovskite-type oxides represented by the general formula (P1).

It is also possible to used, as another material, one of those having perovskite structures which are represented by the following formula (P2):

general formula (Bi₍₁₋ₓ₎Laₓ)FeO₃ (P2)

(where 0<x<1, preferably 0<x≦0.30, and x is La composition in A-site of perovskite structure).

It is also possible to use a material made of a perovskite compound oxide represented by general formula (P3) given below, and including a monoclinic structure.

general formula A(ZnₓTi₍₁₋ₓ₎)_{y}M_{(1-y)}O (P3)

(where A is a Bi element, and M is at least one kind of element selected from the group consisting of Fe, Al, Sc, Mn, Y, Ga, and Yb, x represents a numeric value satisfying 0.4≦x≦0.6, and y represents a numerical value satisfying 0.17≦y≦0.60.)

In addition, it is possible to use a piezoelectric layer having a perovskite-type oxide represented by (NaₓK_{y}Li_{z})NbO₃ (0≦x≦1, 0≦y≦1, 0≦z≦0.2, and x+y+z=1) as a main phase. For example, it is possible to dope sodium potassium niobate (KNN) or sodium potassium lithium niobate with a predetermined amount of Ta (tantalum) or V (vanadium) within the range in which no deterioration occurs in piezoelectric characteristics or surface roughness.

### [Method of Manufacturing Piezoelectric Film]

It is possible to manufacture a piezoelectric film by forming a film containing the constituent elements of a target on a substrate by a chemical vapor deposition method using a plasma with the substrate and the target facing each other. The chemical vapor deposition methods include sputtering methods such as a two-pole sputtering method, three-pole sputtering method, direct current sputtering method, radio frequency sputtering method (RF sputtering method), ECR sputtering method, magnetron sputtering method, opposed target sputtering method, pulse sputtering method, and ion beam sputtering. The present invention can use, as chemical vapor deposition methods, an ion plating method, a plasma CVD method, or the like, in addition to a puttering method.

In the sputtering method, factors that influence the characteristics of a film to be formed include a film formation temperature, the type of substrate, the composition of an underlying film if it is formed beforehand on the substrate, the surface energy of the substrate, a film formation pressure, the amount of oxygen in an atmospheric gas, injection power, the distance between the substrate and the target, the electron temperature in a plasma, an electron density, the active species concentration in the plasma, and the life of active species.

A high-quality film can be formed by optimizing any of, for example, a film formation temperature Ts, Vs−Vf (where Vs is the plasma potential in a plasma at the time of film formation, and Vf is a floating potential), Vs, and a distance D between the substrate and the target. That is, it is possible to form a high-quality film within a given range by plotting the characteristics of a film, with the abscissa representing the film formation temperature Ts, and the ordinate representing any of Vs−Vf, Vs, and the distance D between the substrate and the target.

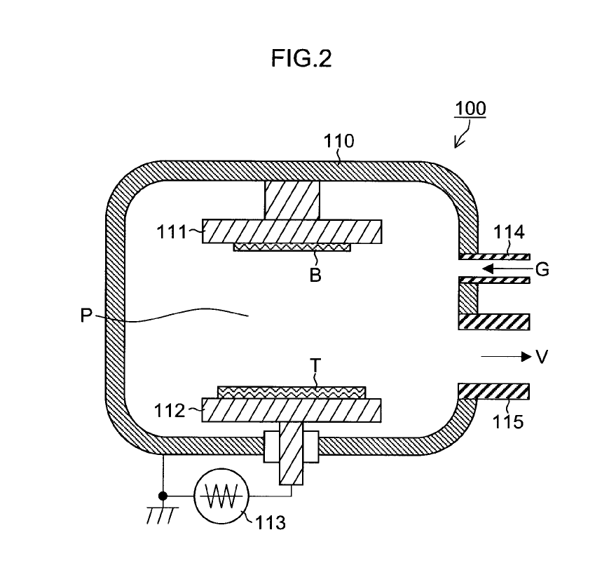

An example of the arrangement of the sputtering apparatus and the manner of film formation will be described with reference to FIG. 2. Although an RF sputtering apparatus using an RF power supply will be described as an example, it is possible to use a DC sputtering apparatus using a DC power supply. FIG. 1 is a schematic sectional view of the overall apparatus.

As shown in FIG. 2, a sputtering apparatus 100 is roughly formed from a vacuum chamber 110 including a substrate holder 111 such as an electrostatic chuck which internally holds the film formation substrate B and can heat the film formation substrate B to a predetermined temperature and a plasma electrode (cathode electrode) 112 which generates a plasma.

The substrate holder 111 and the plasma electrode 112 are spaced apart from each other so as to face each other, and a target T is mounted on the plasma electrode 112. The plasma electrode 112 is connected to an RF power supply 113.

A gas introduction pipe 114 and a gas exhaust pipe 115 are attached to the vacuum chamber 110. The gas introduction pipe 114 serves to introduce a gas G necessary for the formation of a film into the vacuum chamber 110. The gas exhaust pipe 115 serves to perform exhaustion V of the gas in the vacuum chamber 110. As the gas G, for example, Ar or a mixed gas of Ar/O2 is used.

When forming a piezoelectric film of the present invention by a sputtering method, it is possible to form the film while controlling the film formation temperature Ts (° C.) and Vs−Vf (V) representing the difference between the plasma potential Vs (V) and the floating potential Vf (F) during film formation.

### [Performance of Piezoelectric Film]

The performance of the piezoelectric film used by the ultrasonic surgical apparatus 10 of the present invention will be described.

The piezoelectric film formed by a chemical vapor deposition method has a perovskite structure, which is oriented in the (100) or (001) direction, with a degree of orientation of 60% or more. Note that the degree of orientation was obtained by degree of orientation=Σ((peak in (100) direction+peak in (200) direction)/Σ((peak in (100) direction+peak in (200) direction+peak in (110) direction+peak in (111) direction). Note that the peaks in the (100) direction and the (200) direction may be peaks in the direction (001) direction and the (002) direction.

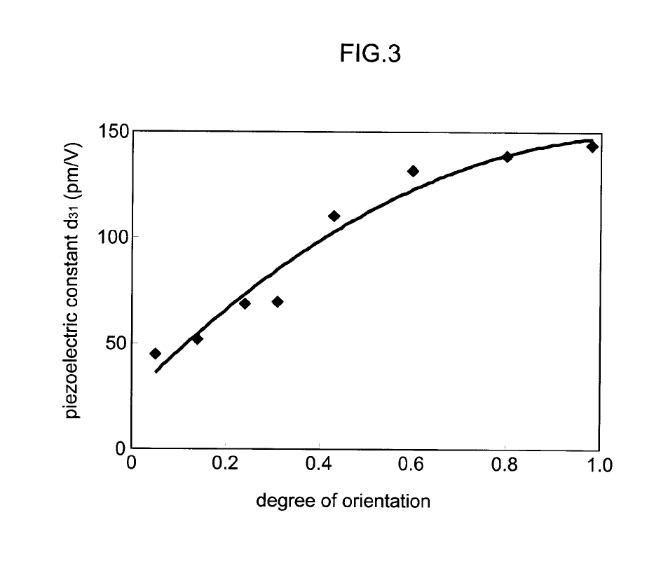

FIG. 3 shows the relationship between the degree of orientation and the piezoelectric constant d₃₁. As shown in FIG. 3, it is possible to increase the piezoelectric constant by increasing the degree of orientation. Since the higher the piezoelectric constant, the lower the driving voltage required for driving operation, it is possible to safely use an ultrasonic surgical apparatus whose driving portion is set in the body like the present invention. It is therefore possible to preferably use the apparatus. The degree of orientation is preferably 60% or more, more preferably 80% or more. Note that in the case of a bulk polycrystalline body, the degree of orientation is about 0.2, and hence the piezoelectric constant d₃₁ is about 91 pm/V.

When the piezoelectric film is used to vibrate the blade portion of the ultrasonic surgical apparatus of the present invention, an increase in driving voltage will lead to noticeable heat generation. Heat generation is proportional to the square of the strength of a magnetic field (E/d), a frequency f, a specific dielectric constant ∈ᵣ, and a dielectric loss tan δ (P₀=(E/d)²×5.56×10⁻¹¹×f×∈ᵣ×tan δ). It is therefore preferable to use a material whose specific dielectric constant ∈ᵣ and dielectric loss tan δ are low. The specific dielectric constant ∈ᵣ of a film oriented in the (100) direction is about 1200, and that in the (001) direction is about 400. It is therefore preferable to use a film oriented in the (001) direction in terms of orientation.

The dielectric loss tan δ varies depending on the quality of a film instead of the orientation of the film. Of films which are different in orientation and equal in tan δ, a film having a higher degree of orientation can be driven at a lower voltage and this is preferable. In the present invention, the dielectric loss is preferably less than 0.4. If the dielectric loss is 0.4 or more, heat is generated at the time of driving, and a sufficient vibration speed cannot be obtained. Setting the dielectric loss to 0.4 or more will require some contrivance such as cooling and intermittent driving. The value of the dielectric loss tan δ is generally the value measured at 1 kHz, which is proportional to heat generation at the time of driving.

According to the present invention, since the piezoelectric film is formed by the chemical vapor deposition method, the film can have a columnar structure, which can be mainly formed from crystal grains of 1 μm or less. Vibrating the columnar structure in the vertical direction will improve the elongation percentage, and hence can improve the durability of the piezoelectric film. A conventional piezoelectric film manufactured by hydrothermal synthesis includes crystal grains of 1 μm or more, and has a randomly oriented structure. With this structure, it is conceivable that the material is displaced in random directions, and hence a larger amount of heat is generated.

In addition, the formed piezoelectric film preferably has a thickness of 10 μm or less. Forming a thick piezoelectric film on the treatment portion will require a voltage higher at the time of driving. For this reason, the piezoelectric film preferably has a thickness of 10 μm or less and drives at a driving voltage of 30 V or less. The driving voltage is preferably 20 V or less, more preferably 15 V or less.

The piezoelectric film formed in this manner can obtain a desired vibration speed at a low driving voltage. It is therefore possible to form a driving portion (piezoelectric film) near the treatment portion to be inserted into the body. This film including a flexible cable extending from the apparatus main body can be inserted into the body while being bent, and hence can be suitably used as a treatment tool such as an endoscope to be inserted into the body.

### Example

A material obtained by processing a 300-μm Ti plate was used as a base material for a blade portion (scalpel portion). As a lower electrode, Ti (20 nm)/Ir (150 nm) was formed on a substrate by the sputtering method. A 5-μm thick PZTN (niobate-lead zirconate titanate) was formed as a piezoelectric film on the lower electrode by the sputtering method. Pt was formed as an upper electrode on the piezoelectric film by patterning.

The obtained scalpel was properly formed without any warpage or any film peeling. In addition, XRD measurement revealed that the piezoelectric film was (100)-oriented on the substrate, and the degree of orientation was 80% or more. In addition, cross-sectional SEM observation revealed that the grain size was 1 μm or less. The dielectric characteristics of the piezoelectric film were represented by E=300 and tan δ=0.3. A PZTN film was separately formed on a Ti substrate under the same conditions, and the piezoelectric constant d₃₁ was measured about 200 pm/V.

When the obtained scalpel was driven, vibration resonance occurred in the longitudinal direction at about 320 kHz. When the scalpel was driven at this frequency and 30 V and the tip was measured by a laser Doppler vibration meter, a vibration speed of 8 msec was obtained. This made it possible to confirm that the scalpel had satisfactory performance.

According to the non-patent literature 1 (Minoru Kurosawa and Takeshi Sasanuma, “Enhancement of Vibration Speed of Micro Ultrasonic Scalpel using PZT Film”, The Institute of Electronics, Technical Report of IEICE, US2009-109 (213) 31) presented as a conventional technique, FIG. 11 shows that a vibration speed of 4 m/s is obtained by forming PZT films on the two surfaces of a 0.3 mm thick Ti metal substrate and driving them at about 20 V. This structure obtains a vibration speed of 7 m/s at about 30 V. In addition, single-surface driving obtains a vibration speed of about 3 m/s at 30 V.

In contrast to this, the piezoelectric film used in the ultrasonic surgical apparatus of the present invention, which has a thickness of 0.3 mm and is formed on a 0.3 mm thick Ti substrate, can obtain a vibration speed of 8 m/s by single-surface driving at 30 V. From practical viewpoints, conventional materials cannot be used for ultrasonic scalpels, whereas using the piezoelectric element of the present invention can form a scalpel which exhibits satisfactory performance at a driving voltage of 30 V. Forming piezoelectric films on the two surfaces of a substrate allows to expect a vibration speed of 8 m/s at a driving voltage of about 15 V. In addition, increasing the transformation ratio by changing the thickness of the horn on the tip allows to expect a higher vibration speed at a lower driving voltage. As has been described above, it was confirmed that the ultrasonic surgical apparatus of the present invention could be safely used in the body at a low driving voltage, and exhibited a considerable improvement in performance as an ultrasonic scalpel.

What is claimed is:

1. An ultrasonic surgical apparatus comprising:
a treatment portion;
a driving portion which drives the treatment portion by resonance;
an operation portion main body which drives and controls the driving portion; and
a connecting portion which connects the treatment portion and the operation portion main body,
wherein the driving portion comprises a piezoelectric film and is formed on the treatment portion inside the connecting portion, and
the piezoelectric film has a perovskite structure, with crystals constituting the perovskite structure being oriented in one of a (100) direction and a (001) direction with a degree of orientation of not less than 60%.

2. The ultrasonic surgical apparatus according to claim 1, wherein the piezoelectric film has a dielectric loss of not more than 0.4.

3. The ultrasonic surgical apparatus according to claim 1, wherein the piezoelectric film has a columnar structure, with crystal grains being not more than 1 μm.

4. The ultrasonic surgical apparatus according to claim 1, wherein the piezoelectric film is formed on the treatment portion through a lower electrode, and the lower electrode and the treatment portion are formed from different materials.

5. The ultrasonic surgical apparatus according to claim 4, wherein the lower electrode comprises a noble metal.

6. The ultrasonic surgical apparatus according to claim 1, wherein the piezoelectric film has a thickness of not more than 10 μm.

7. The ultrasonic surgical apparatus according to claim 1, wherein the piezoelectric film comprises a perovskite-type oxide including one of lead zirconate titanate (PZT) and sodium potassium niobate (KNN).

8. The ultrasonic surgical apparatus according to claim 1, further comprising an upper electrode on the piezoelectric film and a resin which covers the upper electrode or the upper electrode and the piezoelectric film.

9. The ultrasonic surgical apparatus according to claim 1, wherein the piezoelectric films are formed on two surfaces of the treatment portion, and a vibration speed obtained by applying a driving voltage of 15 V to the piezoelectric films is not less than 8 m/s.

10. The ultrasonic surgical apparatus according to claim 1, wherein the treatment portion gradually decreases in width from a portion on which the driving portion is formed to a tip of the treatment portion.
